(19) United States
(12) Patent Application Publication
(10) Pub. No.: US 2005/0049229 A1
(43) Pub. Date: Mar. 3, 2005

(54) Therapeutic applications of 2-hydroxyacetanilide

(76) Inventors: Sheikh Arshad Saeed, Karachi (PK); Shahazad Omar Saeed, (US)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/921,460

(22) Filed: Aug. 19, 2004

(30) Foreign Application Priority Data
Aug. 19, 2003 (GB) 0319477.6

(51) Int. Cl. A61K 31/60; A61K 31/522; A61K 31/485; A61K 31/165; A61K 31/355

(52) U.S. Cl. 514/165; 514/282; 514/406; 514/620; 514/458; 514/474; 514/263.31; 514/570

(57) ABSTRACT

2-Hydroxyacetanilide may be formulated for use in medicine as a therapeutic or prophylactic agent for application as an anti-inflammatory and/or an anti-platelet aggregation drug.

This invention relates to use of 2-hydroxyacetanilide in therapy, in particular as an anti-inflammatory and/or anti-platelet aggregation drug.

2-Hydroxyacetanilide is a commercially available compound (CAS No. 614-80-2) having the formula:

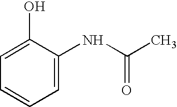

Its positional isomer 4-hydroxyacetanilide, commonly known as paracetamol, is a widely used analgesic and antipyretic agent. Whilst side effects of paracetamol are rare, overdosing may be a problem due to the relatively narrow margin between therapeutic and toxic doses, and can cause severe liver and/or renal damage. As little as 10 g, equivalent to about twenty 500 mg tablets may, for example, cause hepatocellular necrosis.

The corresponding 3-isomer is also known and has been reported to possess antipyretic properties (GB-A-1,006,558) and analgesic properties (U.S. Pat. No. 4,238,508). As far as the applicants are aware, however, 3-hydroxyacetanilide has not to date been incorporated into any regulatory board-approved pharmaceutical compositions.

A study by Dupin et al. (Thrombosis Research, 1988, 50, pp. 437-447) shows that the three positional isomers 2-, 3- and 4-hydroxyacetanilide all exhibit comparable in vitro inhibition of platelet aggregation induced by collagen, arachidonic acid (AA) and adenosine diphosphate (ADP). The authors report that no equivalent in vivo effect is observed with paracetamol but are unable to explain this discrepancy. Their study serves to indicate that in the case of paracetamol, and especially in relation to its anti-platelet aggregation activity, the occurrence of in vitro activity cannot be considered to be an indicator that such activity will also be observed in vivo.

Jiang et al. (Zhonggue Kangshengsu Zazhi, 2000, 25(1), pp. 5-8) report isolation of 2-hydroxyacetanilide from a Streptomyces hygroscopicus culture broth and state that the compound showed a strong inhibitory effect on wild- and mutant-type strains of Saccharomyces cerevisiae.

The present invention is based on the finding that 2-hydroxyacetanilide unexpectedly exhibits valuable activity as both an anti-inflammatory agent and an anti-platelet aggregation. This is particularly surprising given that neither of the corresponding 3- and 4-isomers has been reported to possess such activity in vivo. In the case of paracetamol the absence of these pharmacological activities is generally regarded as an advantage and paracetamol is often the analgesic and/or anti-pyretic drug of choice because of the absence of these properties at normal therapeutic doses.

2-Hydroxyacetanilide is additionally advantageous in that it also exhibits useful levels of analgesic and antipyretic activity and has surprisingly lower toxicity than paracetamol.

Viewed from one aspect the invention provides use of 2-hydroxyacetanilide in the manufacture of a medicament for application as an anti-inflammatory and/or anti-platelet aggregation drug.

Viewed from a further aspect the invention provides a method of treatment of a human or non-human animal subject in need of anti-inflammatory and/or anti-platelet aggregation treatment, said method comprising administering to said subject an effective amount of 2-hydroxyacetanilide.

2-Hydroxyacetanilide for use according to the invention is available commercially (e.g. from Aldrich® or Sigma). Alternatively it may be prepared using standard processes and procedures well known to those skilled in the art.

2-Hydroxyacetanilide for use in accordance with the invention may be formulated as pharmaceutical compositions in any conventional manner with one or more inert carriers, excipients and/or diluents. Examples of suitable carriers, excipients and diluents include water, ethanol, glycerol, polyethylene glycol, sodium chloride, sugars (e.g. glucose, sucrose or lactose), starches (e.g. corn starch or maize starch), microcrystalline cellulose, gums (e.g. gum tragacanth), sorbitol, mannitol, xylitol, magnesium stearate, polyvinylpyrrolidone, fatty acids (e.g. stearic acid), fats, waxes, calcium carbonate, calcium chloride and citric acid.

Such pharmaceutical compositions may additionally comprise one or more wetting agents, sweetening agents (e.g. a sugar, aspartame or saccharin), lubricating agents, emulsifying agents, suspending agents, preserving agents, flavouring agents (e.g. vanillin, peppermint oil or a fruit flavouring) and/or absorption enhancers.

2-Hydroxyacetanilide used in accordance with the invention may if desired be coadministered with further (e.g. one, two or three) pharmaceutically active substances; pharmaceutical compositions comprising 2-hydroxyacetanilide, at least one other pharmaceutically active substance and at least one pharmaceutically acceptable carrier, excipient or diluent comprise a further feature of the invention. An advantage associated with the use of a combination of 2-hydroxyacetanilide and further (e.g. one) active substance(s) is that it may increase the spectrum of diseases for which the composition is suitable for use as a medicament. Additionally or alternatively, a lowering of the dose of 2-hydroxyacetanilide and/or the further active substance(s) may advantageously be achieved. This is particularly advantageous when such a further active substance is associated with known side effects (e.g. as is the case with aspirin).

Additional active substances which may be used in compositions of the invention include non-steroidal anti-inflammatory drugs (NSAIDs), e.g. COX I and/or COX II inhibitors. Representative NSAIDs include aspirin, codeine salts, ibuprofen, mefenamic acid, ketorolac trometamol, indomethacin, celecoxib and COX II inhibitors such as flusulide, NS-398, etodolac or, more preferably, nimesulide.

Other additional active substances which may be present in the compositions of the invention include vitamins (e.g. vitamin C and/or vitamin E), muscle relaxants (e.g. orphenadrine), anti-platelet drugs (e.g. ticlopidine salts or clopidogrel salts) and CNS stimulants (e.g. caffeine).

Particularly preferred compositions of the invention may comprise 2-hydroxyacetanilide and at least one compound (e.g. one or two compounds) selected from aspirin, nimesulide, codeine phosphate, caffeine, orphenadrine, vitamin C and vitamin E. Especially preferred compositions according to the invention may comprise 2-hydroxyacetanilide and one further active substance, preferably a NSAID such as a COX II inhibitor. For example, these compositions may comprise 2-hydroxyacetanilide and aspirin or codeine, particularly aspirin. Alternatively, these compositions may comprise 2-hydroxyacetanilide and nimesulide.

Preferred compositions comprising two or more other active substances include, for example, 2-hydroxyacetanilide, aspirin and another active substance selected from codeine phosphate, caffeine, vitamin C and vitamin E. Examples of such compositions include 2-hydroxyacetanilide, aspirin and caffeine; 2-hydroxyacetanilide, aspirin and codeine phosphate; and 2-hydroxyacetanilide, aspirin, vitamin E and vitamin C.

The selection of additional active substance(s) is preferably made in light of the disease to be treated by administration of the composition. For instance, for application as an anti-inflammatory drug, 2-hydroxyacetanilide may advantageously be formulated together with a NSAID, particularly aspirin and/or codeine (e.g. codeine phosphate). For applications also using its antipyretic properties, 2-hydroxyacetanilide may, for instance, be formulated with vitamin C.

The quantity of 2-hydroxyacetanilide and optional other active substance(s) present in pharmaceutical compositions used in accordance with the invention may be readily determined by those skilled in the art and will depend on several factors, including the nature of any optional further active substance(s), the method of administration, the disease to be treated and the weight of the subject. Generally, however, 2-hydroxyacetanilide may be used in amounts of from 20-80 wt % of the composition, preferably 40-60 wt %. 2-Hydroxyacetanilide is conveniently used in amounts from 30-1500 mg per dosage form, more preferably from 50-500 mg per dosage form, for example, about 250 mg per dosage form. By dosage form is meant per one quantum of medicine taken (e.g. per tablet or per measured amount of liquid).

Any further active substance(s) may, for example, be present in amounts of from 5-70 wt %, preferably 10-40 wt %. An NSAID such as aspirin may, for example, conveniently be used in amounts of from 10-1000 mg per dosage form, preferably from 10-500 mg per dosage form, for example, 200 mg or less per dosage form (suitably about 75-100 mg per dosage form). A COX II inhibitor such as nimesulide may, for example, conveniently be used in amounts of from 50-250 mg per dosage form, preferably from 100-200 mg per dosage form, for example, about 70 mg per dosage form.

Representative quantities of 2-hydroxyacetanilide and further active substances which may be used per dosage form in other compositions of the invention include the following:
2-hydroxyacetanilide (240 mg), aspirin (240 mg) and codeine phosphate (15 mg);
2-hydroxyacetanilide (240 mg), aspirin (240 mg) and caffeine (10-60 mg);
2-hydroxyacetanilide (240 mg) and orphenadrine (100-250 mg)
2-hydroxyacetanilide (10-240 mg), aspirin (10-60 mg), vitamin E (25-50 mg) and vitamin C (100 mg);
2-hydroxyacetanilide (10-300 mg) and ticlopidine hydrochloride (50-500 mg); and

2-hydroxyacetanilide (10-300 mg) and clopidogrel bisulphate (10-250 mg).

Pharmaceutical compositions used in accordance with the invention may be administered orally, rectally (e.g. using a suppository), topically or systemically. The route chosen will depend, for example, on the disease and/or the subject to be treated, although compositions for oral administration are preferred

The compositions may be presented in any form adapted for use in the administration route selected. Forms suitable for oral administration include, for example, plain or coated tablets, sustained release tablets, chewable tablets, soft capsules, hard capsules, suspensions and syrups. Preferred forms for use according to the invention are tablets, suspensions and syrups, particularly tablets.

Forms suitable for systemic administration may, for example, be formulations for intradermal, intraperitoneal or intravenous injection or infusion.

Forms adapted for topical administration include compositions for administration to the skin and mucosa (e.g. gels, creams, sprays, lotions, salves and aerosols).

The compositions are preferably in a ready to use form, although they may also be provided in a concentrate form which is mixed with a liquid (e.g. hot water) immediately prior to use.

Where 2-hydroxyacetanilide is to be used as an anti-inflammatory drug it may, for example, be employed for the therapeutic or prophylactic treatment of muscoskeletal or joint disorders. Such disorders include acute and chronic rheumatic disorders (e.g. arthritis, particularly rheumatoid arthritis, osteoarthritis or juvenile chronic arthritis), spondyloarthropathies (e.g. ankylosing spondylitis, psoriatic arthritis, arthritis associated with inflammatory bowel disorders and arthritis associated with infection), soft tissue rheumatism (e.g. fibromyalgia, humeral epicondylitus (such as tennis or golfer's elbow), frozen shoulder, Tietze's syndrome, fascitus, tendinitus, tensynovitus, bursitus, strains and sprains) and Still's disease.

Where 2-hydroxyacetanilide is alternatively or additionally to be used as an anti-platelet aggregation drug it may, for instance, be employed for the therapeutic or prophylactic treatment of a cardiovascular disorder, by which is meant any condition of the heart, arteries or veins which disrupts the supply of oxygen to life-sustaining areas of the body such as the brain, the heart, etc. Examples of cardiovascular disorders include angina pectoris, stroke, myocardial infarction, arteriosclerosis, arrhythmia and thrombosis (including thrombosis induced by surgery). Preferably, 2-hydroxyacetanilide is used for the prophylactic treatment of cardiovascular disorders (e.g. myocardial infarction).

As noted above, 2-hydroxyacetanilide also exhibits analgesic and antipyretic activity and may thus additionally be used for the therapeutic or prophylactic treatment of pain, including headache (e.g. tension headaches), migraine, dental pain (e.g. toothache), dysmenorrhoea (e.g. period pain), myalgia, back pain (lumbago), rheumatic pain and neuralgia and/or for the therapeutic or prophylactic treatment of fever, including fever induced by infection (e.g. cold and/or influenza symptoms), by neoplastic disease or by drug treatment.

The following non-limiting Examples serve to illustrate the invention:

### EXAMPLE 1

Anti-Inflammatory Action of 2-hydroxyacetanilide

Measurement of Anti-Inflammatory Action of 2-hydroxyacetanilide In Vivo

Paw oedema was induced in rats by injecting 0.1 ml of a freshly prepared 1% suspension of carrageenan in 0.9% NaCl (saline) into the subplanter region of the right hind paws. The plethysmographic method (Winter et al., Proc. Soc. Exp. Biol. Med. III, 1962, pp. 544-547) was then used for recording the volume changes in the rat paw.

In accordance with this method Sprague-Dawley male rats (150-200 g) were divided into seven groups of 8-10 animals each. 2-Hydroxyacetanilide, at doses of 100 or 300 mg kg⁻¹, was injected intraperitoneally into two groups of rats. Another two groups received paracetamol at a dose of 300 or 500 mg kg⁻¹, and a further two groups received aspirin at a dose of 150 or 300 mg kg⁻¹, while the control group received saline or distilled water.

After an interval of 30 minutes the paw volumes were measured and the carrageenan suspension was then injected. The paw volumes were remeasured 5 hours after this injection. The difference in paw volumes before and after the carrageenan injections indicated the volume of inflammation. Anti-inflammatory activities were calculated as A−B/A×100, where A and B denote mean paw volume of control and drug-treated animals respectively. The results are summarised in Table 1.

TABLE 1
Anti-inflammatory effect of 2-hydroxyacetanilide,
paracetamol and aspirin on carrageenan-induced rat-paw
oedema

| | | Mean paw | |
|---|---|---|---|
| | | volume ± SEM | Anti-inflammatory |
| | Dose | after 5 hr | Activity |
| Compound | (mg/kg) | (ml) | Mean ± SEM |
| Control | — | 0.80 ± 0.04 | — |
| 2-hydroxyacetanilide | 100 | 0.52 ± 0.11* | 35 ± 2 |
| 2-hydroxyacetanilide | 300 | 0.24 ± 0.1* | 70 ± 5 |
| Paracetamol | 300 | 0.75 ± 0.06 | 6 ± 2 |
| Paracetamol | 500 | 0.72 ± 0.07 | 10 ± 3 |
| Aspirin | 150 | 0.58 ± 0.08* | 28 ± 9 |
| Aspirin | 500 | 0.23 ± 0.03* | 71 ± 3 |

*p < 0.001 compared with controls
SEM is standard error in mean

The results show that 2-hydroxyacetanilide causes a marked reduction in carrageenan-induced oedema. The mean paw volume observed in the control animals (0.80 ml) was effectively reduced to 0.52 and 0.24 ml after treatment with this compound at doses of 100 and 300 mg kg⁻¹ respectively. The reduction induced by paracetamol was significantly less, even at a higher dose (500 mg kg⁻¹).

### EXAMPLE 2

Anti-Platelet Aggregation Action of 2-hydroxyacetanilide

a) Measurement of Anti-Platelet Aggregation Activity In Vitro

Blood was drawn by vein puncture from healthy human volunteers reported to be free of medications for at least one week. The blood samples were mixed with 3.8% w/v sodium citrate solution (9:1) and centrifuged at 260 g for 15 minutes at 20° C. to obtain platelet rich plasma (PRP). The platelet count was determined by phase contrast microscopy and all aggregation studies were carried out at 37° C. with PRP having platelet counts between 2.5 and 3.0×10⁸/ml of plasma. All experiments were performed within 2 hours of PRP preparation.

Platelet aggregation was monitored using a Dual-channel Lumi-aggregometer (Model 400 Chronolog Corporation, Chicago, USA) using 0.45 ml aliquots of PRP. The final volume was made up to 0.5 ml with the test compound dissolved either in normal saline or an appropriate solvent known to be devoid of any effect on aggregation. Aggregation was induced by arachidonic acid (AA), platelet activating factor (PAF) or collagen.

Anti-platelet aggregation effects were studied by pretreatment of PRP with 2-hydroxyacetanilide or paracetamol for 1 minute followed by the addition of the maximum dose of agonist (i.e. AA, PAF or collagen). The resulting aggregation was monitored for 5 minutes following agonist addition by the change in light transmission as a function of time.

The results were expressed as percentage inhibition after the addition of aggregation agents compared with controls. Once the anti-platelet aggregation activity of the inhibitors against these agonists was established, dose-response curves were constructed to calculate the IC₅₀ values of the inhibitors. The resulting values are shown in Table 2.

TABLE 2
Effect of 2-hydroxyacetanilide and paracetamol on in
vitro platelet aggregation induced by different
aggregation agents (IC₅₀ ± SEM μm)

| | Aggregation Agents | | |
|---|---|---|---|
| Compound | AA | PAF | Collagen |
| 2-Hydroxyacetanilide | 37 ± 0.7 | 87 ± 2 | 209 ± 12 |
| Paracetamol | 136 ± 1 | 167 ± 8 | 263 ± 1 |

2-Hydroxyacetanilide was found to have a significantly lower IC₅₀ value than paracetamol against all the platelet aggregation-inducing agents tested.

b) Measurement of Anti-Platelet Aggregation Activity In Vivo

Both 2-hydroxyacetanilide and paracetamol were tested for in vivo platelet aggregation activity at a dose of 500 mg kg⁻¹. The compounds were first separately dissolved in 0.5% w/v gum tragacanth and then administered orally to albino rabbits of either sex. Control groups received only the vehicle.

Blood was drawn from the marginal ear of the rabbits 2 hours after drug administration. The blood was then mixed with 3.8% w/v sodium citrate solution (9:1) and centrifuged at 260 g for 15 minutes at 20° C. to prepare platelet rich plasma (PRP). The platelet count was determined by phase contrast microscopy and all aggregation studies were carried out at 37° C. with PRP having platelet counts between 2.5 and 3×10′/ml of plasma. The experiments were performed within 2 hours of PRP preparation.

Platelet aggregation was monitored using a Dual-Channel Lumi-aggregometer (Model 400 Chronolog Corporation, Chicago, USA) using 0.45 ml aliquots of PRP. Aggregation was induced with platelet activating factor (PAF, 0.8 μM), collagen (20 μg mL⁻¹) or arachidonic acid (AA, 1.7 mM). The results, expressed as % inhibition based on comparison with the controls, are summarised in Table 3.

TABLE 3
Effect of 2-hydroxyacetanilide and paracetamol on in
vivo platelet aggregation induced by different
aggregation agents (% inhibition)

| | Aggregating Agent | | |
|---|---|---|---|
| Compound | AA | PAF | Collagen |
| 2-Hydroxyacetanilide | 95 ± 3 | 37 ± 2 | 10 ± 2 |
| Paracetamol | 5 ± 3 | 8 ± 2 | — |

The results show that 2-hydroxyacetanilide inhibits both AA and PAF induced aggregation in vivo whereas paracetamol has virtually no anti-platelet aggregatory effect in vivo. The results obtained for paracetamol confirm the observations of Dupin et al. (vide infra) regarding this lack of in vivo activity.

c) Measurement of Prevention of PAF-Induced Mortality in Rabbits

Male rabbits (New Zealand White, 3-3.5 kg) were used to test the ability of 2-hydroxyacetanilide, paracetamol and aspirin to prevent the lethality of intravenous PAF.

PAF was prepared before each injection by dissolution in saline to give a final concentration of 22 μg ml⁻¹. The resulting solution was injected into the marginal ear vein of the rabbits over a period of approximately 1 minute. In preliminary experiments it was established that an intravenous dose of 11 μg kg⁻¹ PAF was required to induce consistently fatal pulmonary thrombosis in the animals (n=6). This dose of PAF was used in subsequent experiments with 2-hydroxyacetanilide and paracetamol.

Rabbits were pretreated by intraperitoneal injection of either 2-hydroxyacetanilide (100 or 300 mg kg⁻¹), paracetamol (300 or 500 mg kg⁻¹) or aspirin (50 mg kg⁻¹) 2 hours before the challenging dose of PAF. Each animal was used for only one experiment. The mortality data is summarised in Table 4.

TABLE 4
Effect of 2-hydroxyacetanilide, paracetamol and aspirin
on PAF-induced mortality in rabbits

| Compound | Dose (mg kg⁻¹) | Route | Survival |
|---|---|---|---|
| PAF-control | 0 | IV | 0/10 |
| 2-Hydroxyacetanilide | 100 | IP | 0/10 |
| 2-Hydroxyacetanilide | 300 | IP | 10/10** |
| Paracetamol | 300 | IP | 0/10 |
| Paracetamol | 500 | IP | 2/10 |
| Aspirin | 50 | IP | 7/10* |

*p < 0.01,
**p < 0.001 compared with control
IP = intraperitoneal,
IV = intravenous

The results show that 2-hydroxyacetanilide at a dose of 300 mg kg⁻¹ effectively prevents PAF-induced death in rabbits whereas paracetamol, at the same dosage level, fails to prevent any deaths. Even at a higher dose of 500 mg kg⁻¹ paracetamol is less effective than the 300 mg kg⁻¹ dose of 2-hydroxyacetanilide.

d) Arachidonic Acid Metabolism by Platelets

Concentrated PRP was centrifuged at 1200 g for 20 minutes and the sedimented platelets were washed twice with ice-cold phosphate buffer (50 mM, pH 7.4) containing NaCl (0.15 M) and EDTA (0.2 mM). After centrifugation the platelets were resuspended in the same buffer, but without the EDTA, at the initial PRP cell concentration. The PRP suspension was homogenized at 4° C. using a Polytron homogenizer for 15 seconds and the homogenate was centrifuged at 1200 g for 20 minutes.

Portions of supernatant (300 μl containing 0.4 mg protein) were incubated with 10 μg unlabelled AA and 0.1 μCi [1-¹⁴C] AA in the presence or absence of test compound. After 15 minutes with gentle shaking in air at 37° C. the reaction was stopped by adding 0.4 ml citric acid (0.4 M) and ethyl acetate (7.0 ml). After mixing and centrifuging at 600 g for 5 minutes at 4° C., the organic layer was separated and evaporated to dryness under nitrogen. The residues were dissolved in 40 μl ethanol and 20 μl was applied to silica gel G thin layer chromatography (TLC) plates (Analtech, Delaware, USA). The AA, thromboxin B2 (TXB₂, a stable degradation product of thromboxin A2 [TXA₂]) and 12-HETE (12-hydroxyeicosatetraenoic acid) standards were spotted separately. The plates were developed in ether/petroleum ether [boiling range 40-60° C.]/acetic acid (50:50:1 v/v) to a distance of 17 cm. By use of this solvent system the various lipoxygenase products (HETEs) are separated with TXB₂ and prostaglandins remaining at the origin. The solvent system used for the separation of various prostaglandins and TXB₂ in dried organic extracts of platelet incubates as above was ethyl acetate/isooctane/water/acetic acid (11:5:10:2 v/v upper phase). Radioactive zones were located and quantified by use of a Berthold TLC linear analyser and chromatography data system (Model LKB 511, Berthold, Germany). Protein concentration was determined by the method of Lowry et al. (J. Biol. Chem., 1951, 193, pp. 265-275) using human serum albumin as standard.

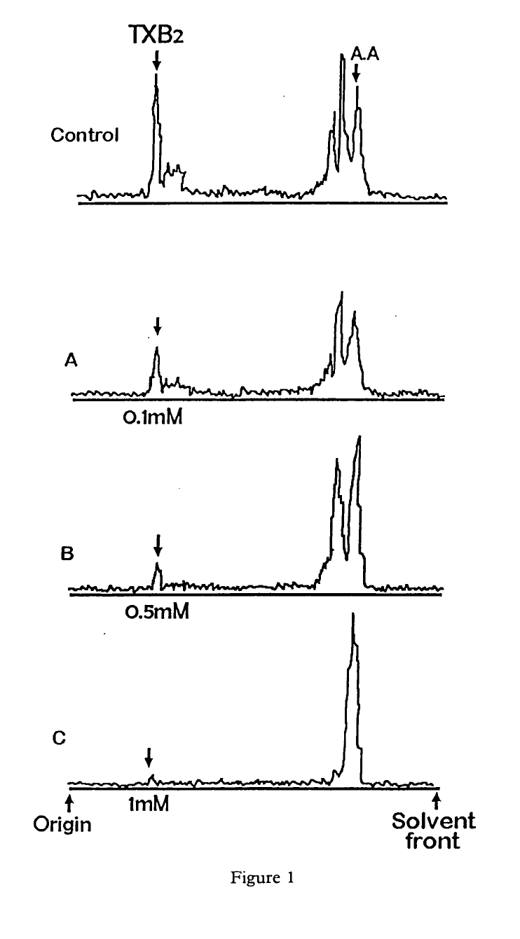

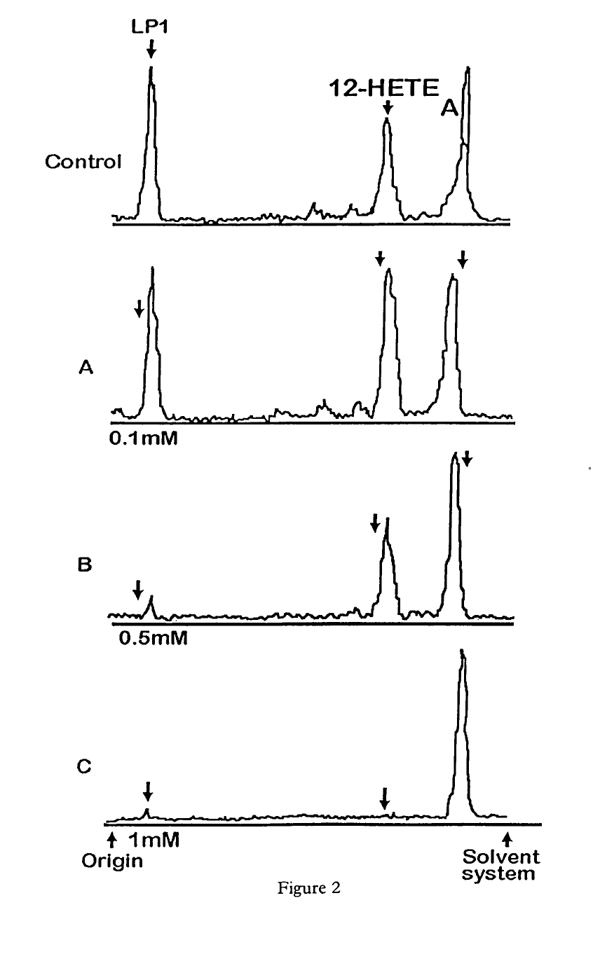

The results are shown in FIGS. 1 and 2. FIG. 1 shows a radiochromatogram of the products of cyclo-oxygenase metabolites of AA formed upon incubation of homogenate of human platelets with [1-¹⁴C] AA in the absence and presence of 2-hydroxyacetanilide. It can be seen from FIG. 1 that with 1 mM 2-hydroxyacetanilide no cyclooxygenase metabolites are formed; that is that AA-induced enzyme activity, which is involved in platelet aggregation, is effectively inhibited by 2-hydroxyacetanilide.

FIG. 2 shows a radiochromatogram of the profile of arachidonate lipoxygenase 1 (LP1) metabolites produced by an homogenate of human platelets incubated with [1-¹⁴C]AA in the absence and presence of 2-hydroxyacetanilide. It can be seen from FIG. 2 that 2-hydroxyacetanilide also inhibits the arachidonate lipoxygenase enzyme since at a concentration of 1 mM virtually no metabolites are formed.

### EXAMPLE 3

Measurement of COX II Inhibition by 2-hydroxyacetanilide

COX II-containing tissues were homogenized at 4° C. in Tris Buffer (50 mM, pH 7.4) containing phenylmethylsulfonyl fluoride (1 mM), pepstatin A (1.5 mM) and leupeptin (0.2 mM) in a ratio of 5:1 (v/w). The protein concentration in the homogenates was measured using the Bradford assay (Anal. Biochem. 1976, 72, p. 248) adapted for a 96-well plate reader, using bovine serum albumin as standard. Homogenates were incubated at 37° C. for 30 minutes in the presence of excess arachidonic acid (30 μM). The samples were boiled and centrifuged at 10,000×g for 30 minutes. The concentration of 6-keto PGF₁, or PGE₂ present (which is indicative of COX II activity) in the supernatant was measured by radio immunoassay (radiolabeled prostanoids were obtained from Amersham; antibodies were from Sigma). The results are shown in Table 5:

TABLE 5
Inhibition of COX II by 2-hydroxyacetanilide

| Concentration of 2- | Percentage Inhibition of |
|---|---|
| hydroxyacetanilide (μM) | PGE₂ production |
| 1 | 21 ± 4 |
| 10 | 52 ± 3 |
| 100 | 90 ± 4 |

The results show that at a concentration of 100 μM 2-hydroxyacetanilide effectively inhibits the COX II enzyme. This is clear from the fact that at this concentration the production of PGE₂ by COX II is inhibited.

### EXAMPLE 4

Analgesic Activity of 2-hydroxyacetanilide

Measurement of Analgesic Activity In Vivo—Acetic Acid Induced Writhing Test in Mice

Analgesia was assessed by the method of Koster et al. (Federation Proceedings, 1959, 18, pp. 412-416), otherwise known as the acetic acid induced writhing test. When acetic acid is injected intraperitoneally it causes abdominal contractions or writhings, a syndrome characterized by a wave of contraction of the abdominal musculature followed by extension of hind limbs. By counting the number of writhes a measure of the effective analgesia provided can be obtained.

Male Wister mice (20-25 g) were divided into six groups, with five mice per group, for use in the writhing assay. The compounds 2-hydroxyacetanilide or paracetamol were administered orally 30 minutes prior to an intraperitoneal injection of 1% acetic acid. Immediately after administering the acetic acid, the number of writhings were counted and recorded for the subsequent 10 minutes. Analgesic potency was taken as difference between a test group mean and its control. The results are shown in Table 6.

TABLE 6
Analgesic effects of 2-hydroxyacetanilide and
paracetamol on acetic acid induced writhing in mice

| | | | No. of | |
|---|---|---|---|---|
| Compound | Dose (mg kg⁻¹) | Route | writhings | % Inhibition |
| Control | 10 | IP | 30 | — |
| (Acetic Acid) | | | | |
| 2-Hydroxyacetanilide | 100 | oral | 27 | 10 |
| 2-Hydroxyacetanilide | 300 | oral | 11 | 64 |
| 2-Hydroxyacetanilide | 500 | oral | 4 | 87 |
| Paracetamol | 300 | oral | 20 | 33 |
| Paracetamol | 500 | oral | 3.2 | 89 |

IP = intraperitoneal

The results show that 2-hydroxyacetanilide is more effective per unit than paracetamol in inhibiting analgesia induced by acetic acid injection. At a dose of 300 mg/kg, 2-hydroxyacetanilide inhibited 64% of writhes whereas, at the same dose, paracetamol only inhibited 33% of writhes. This illustrates that 2-hydroxyacetanilide may be used at lower concentrations than paracetamol whilst providing the same amount of analgesia.

1. A method of treatment of a human or non-human animal subject in need of anti-inflammatory and/or anti-platelet aggregation treatment, said method comprising administering to said subject an effective amount of a pharmaceutical composition comprising 2-hydroxyacetanilide.

2. A method as claimed in claim 1 wherein said composition contains at least one other pharmaceutically active substance.

3. A method as claimed in claim 2 wherein said at least one other pharmaceutically active substance is an NSAID or a COX II inhibitor.

4. A method as claimed in claim 3 wherein said NSAID is selected from aspirin, codeine salts, ibuprofen, mefenamic acid, ketorolac trometamol, indomethacin and celecoxib.

5. A method as claimed in claim 4 wherein said NSAID is aspirin.

6. A method as claimed in claim 3 wherein said COX II inhibitor is nimesulide.

7. A method as claimed in claim 2 wherein said at least one other pharmaceutically active substance is selected from caffeine, codeine phosphate, vitamin C and vitamin E.

8. A method as claimed in claim 2 wherein said at least one other pharmaceutically active substance is orphenadrine.

9. A method as claimed in claim 2 wherein said at least one other pharmaceutically active substance is ticlopidine hydrochloride or clopidogrel bisulphate.

10. A pharmaceutical composition comprising 2-hydroxyacetanilide together with at least one other pharmaceutically active substance and at least one pharmaceutically acceptable carrier, excipient or diluent.

11. A composition as claimed in claim 10 wherein said at least one other pharmaceutically active substance is an NSAID or a COX II inhibitor.

12. A composition as claimed in claim 11 wherein said NSAID is selected from aspirin, codeine salts, ibuprofen, mefenamic acid, ketorolac trometamol, indomethacin and celecoxib.

13. A composition as claimed in claim 12 wherein said NSAID is aspirin.

14. A composition as claimed in claim 11 wherein said COX II inhibitor is nimesulide.

15. A composition as claimed in claim 10 wherein said at least one other pharmaceutically active substance is selected from caffeine, codeine phosphate, vitamin C and vitamin E.

16. A composition as claimed in claim 10 wherein said at least one other pharmaceutically active substance is orphenadrine.

17. A composition as claimed in claim 10 wherein said at least one other pharmaceutically active substance is ticlopidine hydrochloride or clopidogrel bisulphate.
